(19) United States
(12) Patent Application Publication
(10) Pub. No.: US 2024/0200110 A1
(43) Pub. Date: Jun. 20, 2024

(54) METHODS AND ARRANGEMENTS FOR CONTROLLING ENZYMATIC HYDROLYSIS BY FTIR SPECTROMETRY

(71) Applicant: UPM-KYMMENE CORPORATION, Helsinki (FI)

(72) Inventors: Vilho NISSINEN, Vehkataipale (FI); Meri VENTOLA, Leipzig (DE); Juha TAMPER, Levänen (FI); Sami TURUNEN, Lappeenranta (FI); Juha SEPPÄ-LASSILA, Lappeenranta (FI)

(21) Appl. No.: 18/287,231

(22) PCT Filed: Jun. 4, 2021
(86) PCT No.: PCT/FI2021/050417

§ 371 Date: Oct. 17, 2023

(51) Int. Cl. C12P 19/02; C12M 1/34; C12M 1/36; C12P 7/18; C12P 19/14

(52) U.S. Cl. CPC C12P 19/02; C12M 41/30; C12M 41/48; C12P 7/18; C12P 19/14

(57) ABSTRACT

A method is provided for controlling enzymatic hydrolysis in a manufacturing process of chemical bioproducts. The method comprises performing at least one FTIR measurement on at least one process fluid, and controlling the value of at least one process parameter based on the results obtained. Said results indicate the content of one or more carbohydrates in the respective process fluid. Said controlling of the value of the process parameter is performed in order to affect at least one of: the conversion of cellulose and hemicellulose into monomeric carbohydrates in said enzymatic hydrolysis, the relative content of soluble lignin in relation to monomeric carbohydrates in said enzymatic hydrolysis.

### TECHNICAL FIELD

The disclosure relates in general to controlling an industrial-scale manufacturing process of chemical bioproducts. In particular, the disclosure relates to the application of specific measurement methods at various parts of the process and to the control decisions that can be made on the basis of such measurements.

### BACKGROUND OF THE INVENTION

The production of biomass-based chemicals may use for example wood particles as the main raw material. In a biomass-to-sugar process the wood particles or other biomass may be subjected to various kinds of pretreatment such as washing and impregnating with water, acid catalyst, and/or other liquids, and subjected to elevated temperature and pressure, in order to prepare the material for later steps of the process. The later steps may involve for example enzymatic hydrolysis, from which the sugars (carbohydrates) may be fed further to other processes. Such other process may involve the production of for example glycols. The enzymatic hydrolysis step may also produce lignin as one of its outputs.

In its known form, controlling the enzymatic hydrolysis step involves a number of uncertainties. The process may be designed for certain nominal enzyme loading and activity, but how accurately these enable achieving a target level of glucose content in a given time may depend on e.g. how successful the preceding pretreatment step was in preparing the material flow. It would be advantageous to have a possibility to react in real time (or at least as quickly as possible) to detected deviations from the expected proceeding of the process. However, it is difficult to obtain accurate knowledge of the current status of each step in the process in real time. Any measurement method that is to be applied must be applicable for prolonged operation in the harsh conditions of an industrial environment, which typically makes it difficult or impossible to utilize instruments built for use in laboratory conditions.

### SUMMARY

According to a first aspect there is provided a method for controlling enzymatic hydrolysis in a manufacturing process of chemical bioproducts. The method comprises performing at least one Fourier Transform Infrared (FTIR) measurement on at least one process fluid of said manufacturing process, and controlling the value of at least one process parameter based on the results obtained from said at least one FTIR measurement. Said results indicate the content of one or more carbohydrates in the respective process fluid. Said controlling of the value of the process parameter is performed in order to affect at least one of: the conversion of cellulose and hemicellulose into monomeric carbohydrates in said enzymatic hydrolysis, the relative content of soluble lignin in relation to monomeric carbohydrates in said enzymatic hydrolysis.

In the context of this text, cellulose is taken to mean at least one or even all of: fibers, fiber particles, cellulose, glucane, oligomeric glucose. In the context of this text, hemicellulose is taken to mean at least one or even all of: xylan (like glucuronoxylan and arabinoxylan), xylooligomers, other hemicellulosic oligomeric sugars.

According to an embodiment, the method comprises performing an FTIR measurement on the contents of an enzymatic hydrolysis reactor where said enzymatic hydrolysis is currently taking place. This involves the advantage that the proceeding of the enzymatic hydrolysis reaction can be followed essentially in real time.

According to an embodiment, the method comprises taking a sample of the contents of said enzymatic hydrolysis reactor and conveying said sample into an FTIR measurement point for performing said FTIR measurement. This involves the advantage that it is not necessary to build the FTIR measurement capability directly into the enzymatic hydrolysis reactor, which makes its structural design simpler and may facilitate easier maintenance of the FTIR measurement apparatus.

According to an embodiment, the method comprises performing an FTIR measurement on the contents of a process stream immediately downstream of said enzymatic hydrolysis reactor. This involves the advantage that one can obtain accurate indications of how the enzymatic hydrolysis reaction succeeded.

According to an embodiment, the manufacturing process comprises a separation step downstream of said enzymatic hydrolysis reactor for separating solids from liquids, and the method comprises performing an FTIR measurement on a liquid output of said separation step. This involves the advantage that one may use the results of the FTIR not only to making conclusions about the enzymatic hydrolysis reaction but also to monitor how well the collection of the produced monomeric carbohydrates succeeds.

According to an embodiment, the method comprises controllably conveying samples taken from a plurality of sampling points along said manufacturing process into a common FTIR measurement point in a time-divided manner, and performing FTIR measurements of said plurality of samples sequentially at said FTIR measurement point. This involves the advantage that a single FTIR measurement apparatus can be used to perform FTIR measurements for monitoring a number of steps in the process.

According to an embodiment, the controlling of the value of the process parameter comprises controlling the dosing of at least one enzyme into said enzymatic hydrolysis. This involves the advantage that the utilization of relatively expensive process chemicals can be optimised.

According to an embodiment, the controlling of the value of the process parameter comprises controlling the residence time of the processed product in said enzymatic hydrolysis reactor. This involves the advantage that the operation of the process can be controlled with relatively simple means.

According to an embodiment, the enzymatic hydrolysis is made in consecutive product batches in the process. The controlling of the value of the process parameter may then comprise controlling the efficiency of intermediate cleaning in preparation of a subsequent product batch in said manufacturing process. This involves the advantage that the disadvantageous effects of contamination can be mitigated in time, with measures that are appropriately dimensioned for each individual occasion.

According to an embodiment, the results of the FTIR measurement are obtained by calculating, for a plurality of instants of time, a respective weighted linear combination of FTIR-measured absorbance values at selected wave numbers and using the calculated weighted linear combination as an indicator of measured concentration of a monomeric carbohydrate at each such instant of time. This involves the advantage that a number of aspects that affect the FTIR measurement can be taken into account.

According to an embodiment, said selected wave numbers include at least one compensatory wave number selected for temperature compensation. Absorbance at said compensatory wave number is less sensitive to the concentration of said monomeric carbohydrate than absorbances at others of said selected wave numbers. This involves the advantage that temperature-induced inaccuracies can be mitigated from the measurement without having to measure the temperature with any additional instruments.

According to a second aspect, there is provided an arrangement for controlling enzymatic hydrolysis in a manufacturing process of chemical bioproducts. The arrangement comprises at least one reactor for subjecting a process stream of said manufacturing process to enzymatic hydrolysis, as well as additional processing equipment upstream and downstream of said reactor in the process. The arrangement comprises at least one Fourier Transform Infrared (FTIR) measurement station configured to measure the content of one or more carbohydrates in a process fluid contained in said reactor or in any of said additional processing equipment. The arrangement comprises also a process controller coupled to receive measurement results from said at least one FTIR measurement station. Said process controller is configured to control the value of at least one process parameter of the manufacturing process at least partly on the basis of the received measurement results in order to affect the conversion of cellulose and hemicellulose into monomeric carbohydrates in said enzymatic hydrolysis and/or the relative content of soluble lignin in relation to monomeric carbohydrates in said enzymatic hydrolysis.

According to an embodiment, the arrangement comprises a plurality of FTIR measurement stations, each configured to measure the content of the respective carbohydrate or carbohydrates in a respective process fluid. This involves the advantage that real-time FTIR measurement data can be obtained from various steps of the process at any desired time.

According to an embodiment, the arrangement comprises a common FTIR measurement station, so that said fluid handling means are configured to controllably convey samples taken from a plurality of sampling points along said manufacturing process into said common FTIR measurement point in a time-divided manner. This involves the advantage that a single FTIR measurement apparatus can be used to perform FTIR measurements for monitoring a number of steps in the process.

According to a third aspect, there is provided the use of a Fourier Transform Infrared (FTIR) measurement, made on at least one process fluid of a manufacturing process of chemical bioproducts, to control the value of a process parameter based on the results obtained from said FTIR measurement. Said results indicate the content of one or more carbohydrates in the respective process fluid. Said controlling of the value of the process parameter is performed in order to affect the conversion of cellulose and hemicellulose into monomeric carbohydrates in said enzymatic hydrolysis and/or the relative content of soluble lignin in relation to monomeric carbohydrates in said enzymatic hydrolysis.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 is a high-level block diagram of a manufacturing process of chemical bioproducts,

FIG. 2 illustrates the process steps of an exemplary enzymatic hydrolysis process,

FIG. 3 illustrates possible ways of applying an FTIR measurement,

FIG. 4 illustrates the possible multiplexing of samples to an FTIR measurement,

FIG. 5 illustrates the effect of enzyme loading or activity,

FIG. 6 illustrates the effect of contamination,

FIG. 7 illustrates the effect of contamination in a later hydrolysis step,

FIG. 8 illustrates an example of variation in yield between batches,

FIG. 9 illustrates an example of a declining yield in a series of batches,

FIG. 10 illustrates an example of an FTIR measurement,

FIG. 11 illustrates an example of an FTIR measurement,

FIG. 12 illustrates an example of an FTIR measurement, and

FIG. 13 illustrates a comparison between FTIR-based analysis and laboratory measurements.

### DETAILED DESCRIPTION

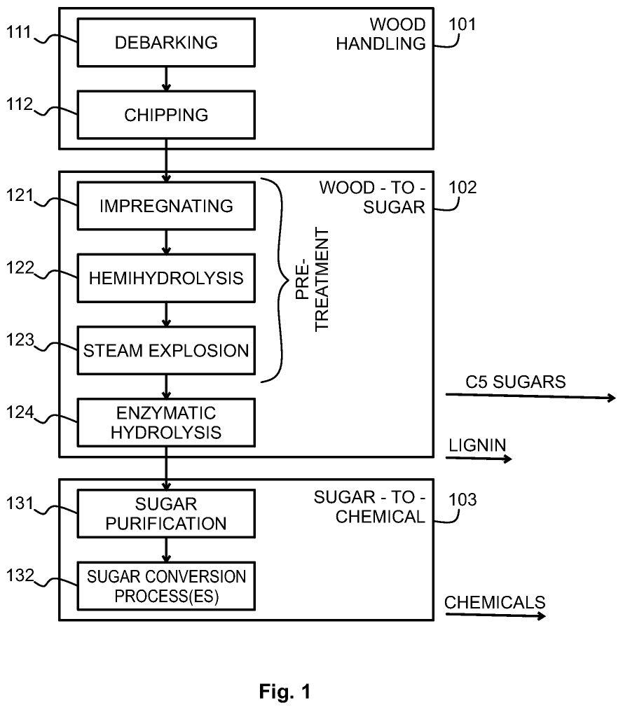

FIG. 1 illustrates schematically a manufacturing process of chemical bioproducts from wood material. The process can be roughly divided into a wood handling phase 101, a wood-to-sugar phase 102, and a sugar-to-chemical phase 103. The wood material may be selected from a group consisting of hardwood, softwood, and their combination. The wood material may e.g. originate from pine, poplar, beech, aspen, spruce, or birch. The wood material may also be any combination or mixture of these. Preferably the wood material is broadleaf wood due to its relatively high inherent sugar content, but the use of other kinds of wood is not excluded.

The wood handling phase 101 comprises mainly mechanical processing such as debarking 111 and chipping 112.

The wood-to-sugar phase 102, which is also called the wood-to-sugar process, comprises a pre-treatment part where the wood chips from the wood handling phase 101 are taken through impregnating 121, hemihydrolysis 122, and steam explosion 123 in order to break down the structure of the wood material and to remove the C5 sugars. Impregnating is typically part of processes that utilize an acid catalyst, so it may be omitted in processes that rely upon autohydrolysis. The main process stream continues into enzymatic hydrolysis 124, where the aim is to convert polysaccharides into C6 monomers, essentially converting glucan into glucose. Lignin and other remaining solids are removed after the enzymatic hydrolysis, and the obtained C6 sugars are fed further to a sugar-to-chemical phase 103. The removed lignin may be utilized further in other processes.

The subsequent utilization of the sugars in the sugar-to-chemical phase 103 may comprise steps such as purification 131 of the sugars (both C5 and/or C6 carbohydrates) and one or several sugar conversion processes 132. The sugar conversion processes 132 may include processes such as fermentation to produce alcohols or catalytical hydrotreatment to produce glycols.

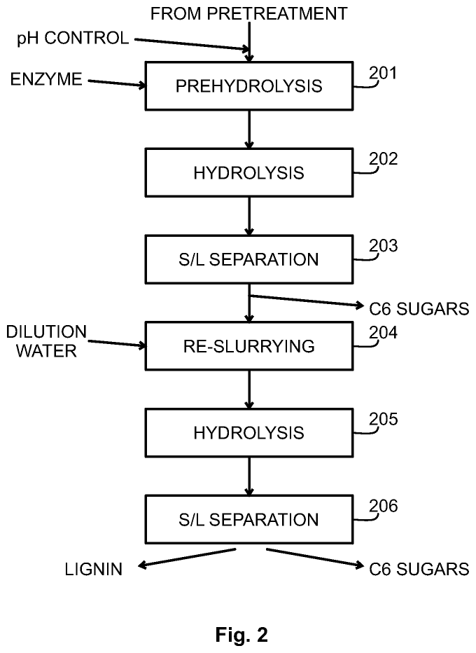

FIG. 2 illustrates in more detail an example of what may be included in that part of the process that in FIG. 1 was only represented by the enzymatic hydrolysis step 124. The process stream that comes from the pretreatment part has the form of a water-based slurry. It contains mainly cellulose, but also small amounts of hemicellulose. One purpose of the preceding pretreatment part was to remove hemicellulose and C5 sugars, but some always remains. The enzymatic hydrolysis step is mainly designed to convert the cellulose into monomeric carbohydrates (C6 carbohydrates), but at the same time it also serves to convert the small remaining fraction of hemicellulose into respective monomeric carbohydrates (C5 carbohydrates).

The slurry may be subjected to some pH control, after which it goes into a short prehydrolysis 201 where selected enzymes are added. The subsequent first hydrolysis step 202 is preferably made in batches, so that the conditions and proceeding of the hydrolysis reaction can be monitored and controlled.

A filtrate from the first solid/liquid separation step 203 brings out some of the soluble C6 carbohydrates already, while the solid fraction is taken to re-slurrying 204 and further to an additional (second) hydrolysis step 205.

In this text, re-slurrying means a method step (and the corresponding processing equipment) in which a processed product is made more fluent, typically by adding water or some water-based solution. In a process like those described here, re-slurrying is frequently used after solid/liquid separation, in order to make the separated solid fraction easier to handle and also in order to further clean it from any remaining soluble compounds in a subsequent further solid/liquid separation step.

The output from the second enzymatic hydrolysis step 205 is led to a second solid/liquid separation step 206, where a liquid fraction comprising C6 carbohydrates and a solid fraction are separated from each other. There may be consecutive rounds of solid/liquid separation and re-slurring, and the number of such consecutive rounds may vary. The separated lignin comes out of the final separation step.

Of key importance to the efficient production of C6 carbohydrates is the successful conversion from glucane in the prehydrolysis 201 and hydrolysis steps 202 and 205. Factors that affect the efficiency of the hydrolysis include—but are not limited to—the following: the extent to which the preceding pretreatment and hemihydrolysis achieved their desired results; the selection and dosing of the enzyme(s); the pH and temperature of the slurry in which the hydrolysis takes place; the efficiency of mixing the slurry during the reaction period; the possible presence and constitution of chemical inhibitors like organic acids or furans; the possible presence and nature of microbial contamination; and even the tree species or other nature of the original source of raw material. While the effect of many such factors can be predicted at least to some extent and acted upon, it would be highly advantageous to be able to monitor in real time (or, at least, with as short a delay as possible) how the conversion proceeds. What is here said about the conversion of cellulose into C6 carbohydrates applies also to the conversion of hemicellulose into C5 carbohydrates: the conversion reactions behave in a suitably similar manner, so that actions taken to optimize the conversion of cellulose into C6 carbohydrates are likely to have an advantageous effect also for the conversion of (the small amount of) hemicellulose into C5 carbohydrates.

In order to have a better control over the successful obtaining of the desired end products from a manufacturing process of chemical bioproducts like those described above, there has been developed a novel method for controlling the enzymatic hydrolysis.

An element of the method is the performing of at least one FTIR measurement on at least one process fluid of the manufacturing process. The acronym FTIR comes from Fourier Transform InfraRed, which means a spectroscopic measurement method in which a sample is subjected to a beam of radiation that covers a wide band in the infrared region of wavelengths. A set of high-resolution spectral data is collected in order to examine, how much of the incident infrared radiation becomes absorbed in the sample substance. The collected data, also referred to as the interferogram, is subjected to mathematical processing that has the nature of a Fourier transform. As a result, it gives a spectrum indicative of the relative absorption of different wavelengths of infrared radiation in the sample. As different chemicals give rise to different kinds of absorption, the calculated spectrum is a kind of spectral fingerprint of the actual chemical constitution of the measured sample.

There are other kinds of spectroscopic measurements in the infrared region that are known from chemical wood processing industry, such as NIR (meaning Near InfraRed). However, unlike NIR which typically requires a solid sample and produces measurement results mostly indicative of the solid constituents in the sample, FTIR is applicable for directly measuring fluid samples and produces results indicative of the chemical constituents in the liquid phase. On the other hand, known FTIR measurement methods have a relatively short penetration depth in the fluid sample. In slurries such as those encountered in enzymatic hydrolysis, the penetration depth is close to zero. This is because the measurable interaction between the incident radiation and the measured sample takes place at (or at least very close to) the outer surface of the outermost optical element, typically a diamond crystal, through which the radiation is conveyed towards the sample.

The method of controlling enzymatic hydrolysis described here involves controlling the value of at least one process parameter based on the results that are obtained from the at least FTIR measurement. As the measurement is performed on at least one process fluid, the results indicate the content of one or more carbohydrates in the respective process fluid. In one embodiment, such carbohydrates may be described as sugars of interest, where the term sugars is used to mean any mono- or polysaccharide the relative amount of which in the measured process fluid enables making deductions about how the enzymatic hydrolysis proceeds or has succeeded. For example, a carbohydrate or sugar of interest in the FTIR measurement may be one of: glucan, its conversion product glucose; xylan, its conversion product xylose; mannan, its conversion product mannose; arabinoxylan, its conversion product arabinose; lactam, its conversion product lactose. The controlling of the value of a process parameter is performed in order to affect the conversion of cellulose and hemicellulose into monomeric carbohydrates (for example glucan into glucose, and/or xylan into xylose) in the enzymatic hydrolysis and/or the relative content of soluble lignin in relation to monomeric carbohydrates in the enzymatic hydrolysis.

In case an additional method step is needed to obtain accurate measurements of soluble lignin, such an additional method step may comprise a spectrophotometric absorbance measurement, for example at a wavelength 205 nanometres. In a detailed example of such a method, a 10 ml sample is taken from the solution after enzymatic hydrolysis. If the sample is turbid or cloudy, the sample is diluted with high purity (distilled or deionized) water and filtrated. Absorbance is measured with an UV-spectrophotometer at the wavelength 205 nm; 1 cm cuvettes are used in the measurement. If the absorption is over 0.7 AU, the sample is diluted with high purity water until the absorption is in the range 0.2-0.7 AU. The zero value is measured by putting high purity water in a cuvette and measuring the water sample as a blank sample or reference. Two parallel measurements of the sample are taken to verify the results. This measurement method is based on the difference in absorption between soluble lignin in water solution and blank solution, which is water. By subtracting the spectrum derived from the blank solution from that of the lignin solution, an absorbance difference is obtained. The amount of soluble lignin may then be calculated by using the following equation. Possible dilutions are taken into account in the calculations. The results are reported as integers and the unit is mg/l.

Calculating the amount of soluble lignin (mg/i):

x=(A/a)×D

where
A=Absorbance
a=Absorbtivity coefficient 0,110 l/mgcm,
D=Dilution factor.

Absorptivity coefficient 110 l/gcm (notice the unit) is used as an average for samples which contain different wood species.

If the enzymatic hydrolysis proceeds as intended, the absolute amount of lignin in the slurry remains constant but the relative amount of monosaccharides such as glucose increases as the conversion proceeds. Only a very small amount of the lignin is soluble, as long as the pH of the slurry remains smaller than about 5.5. Dissolving lignin into the liquid phase weakens the quality of the sugar that is the desired end product, so typically it should be avoided. With the enzymes known at the time of writing this description, a desirable pH range of the slurry in enzymatic hydrolysis is about 4-5.5. Even lower pH values, like pH=3 for example, would be even more desirable, because they might help in preventing microbial contamination. However, it is not easy to find enzymes that would work properly in the described process with pH values of the slurry smaller than 4.

An advantageous range of infrared wave numbers to be used in the FTIR measurement is 648-4000 l/cm. By performing calibration measurements in laboratory, it is possible to identify “signature features” in the FTIR spectrum that are indicative of e.g. the relative concentration of one or more monosaccharides such as glucose in the measured sample. Additionally or alternatively, it is possible to calculate the relative (remaining) concentration of one or more polysachharides such as glucane. Additionally or alternatively, it is possible to identify features of the FTIR spectrum that are solely indicative of the relative content of (soluble) lignin in relation to monomeric carbohydrates in the measured sample. While the relative amount of soluble lignin is small, it is nevertheless there so its fingerprint in the FTIR spectrum can be utilized.

Even features of the FTIR spectrum that as such cannot be unambiguously associated with any particular constituent in the measured process fluid may be of importance. Namely, there may be “standard” or “routine” forms of the FTIR spectrum that are routinely observed when the enzymatic hydrolysis proceeds as intended. If a “mystery” feature like unexpected absorption at some sub-range of wavelengths appears, and/or if there is observed a trend-wise change in the FTIR spectrum or part of it that has not been encountered before, it typically tells that the enzymatic hydrolysis is currently not proceeding as it should so it may be taken as an alert to e.g. measure the amount of contamination or perform some other observational or correctional measures.

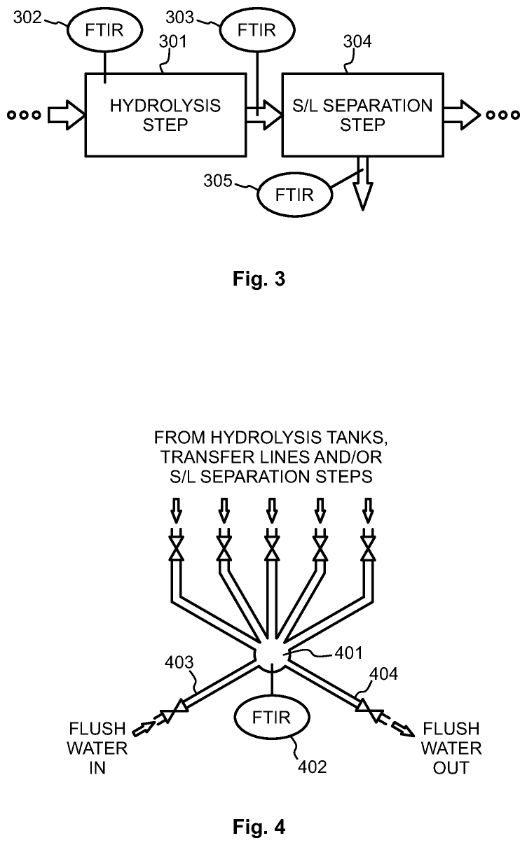

FIG. 3 illustrates some locations in the process where process fluids suitable for the FTIR measurement appear and where an FTIR measurement may consequently produce useful information.

As a process step, the enzymatic hydrolysis 301 may be performed in batches or as a continuous process. Assuming the first-mentioned, the method for controlling enzymatic hydrolysis may comprise performing an FTIR measurement 302 on the contents of an enzymatic hydrolysis reactor where the enzymatic hydrolysis 301 is currently taking place.

Several alternatives exist for performing such an FTIR measurement 302. It is possible to build the enzymatic hydrolysis reactor so that it contains a built-in measurement head for the FTIR measurement. In order to get reliable results that represent well the current contents of the reactor, it is advisable to place such a built-in measurement head so that there is sufficient turbulence of the slurry contained in the reactor at the location of the measurement head. Such a measurement head may e.g. protrude into the reactor from the inside wall of the reactor by a distance of 0 to 20 cm, preferably 1 to 5 cm. If there is a mixing device in the reactor, such as a picket-fence stirrer for example, an advantageous location for the built-in measurement head may be one where a blade end of the stirrer makes repeated sweeps adjacent to the measurement head. Placing the measurement head within a recess or hollow is not advisable, because if such forms exist in the enzymatic hydrolysis reactor they tend to significantly slow down the mixing of the portion of the slurry contained in them with the main part of the slurry. It has been found that if the mixing of the slurry in an enzymatic hydrolysis reactor is not efficient, it may take as long as half an hour before a change made by e.g. adding some more enzyme or pH stabilizer takes effect truly throughout the whole reactor.

Another alternative way of performing an FTIR measurement 302 on the contents of an enzymatic hydrolysis reactor is one that involves taking a sample of the contents of the enzymatic hydrolysis reactor and conveying the sample into an FTIR measurement point for performing the FTIR measurement. This alternative is particularly advantageous, if there is a centralized FTIR measurement point where samples taken from different parts of the process may be conveyed to for measurement.

Performing the FTIR measurement on the contents of an enzymatic hydrolysis reactor involves the advantage that the proceeding of the hydrolysis reaction can be followed continuously or at least repeatedly during the time the batch of slurry resides in the reactor.

As the manufacturing process of chemical bioproducts may comprise two or more prehydrolysis and enzymatic hydrolysis steps (see e.g. steps 201, 202, and 205 in FIG. 2), it should be noted that the step 301 shown in FIG. 3 may be any of these, most advantageously any or both of the enzymatic hydrolysis steps 202 or 205. In other words, the performing of an FTIR measurement on the contents of an enzymatic hydrolysis reactor may mean performing the measurement on the contents of any of the enzymatic hydrolysis reactors in the process, or in any combination of these.

Reference designator 303 in FIG. 3 illustrates how, as an addition or alternative to the FTIR measurement 302, the method may comprise performing an FTIR measurement on the contents of a process stream immediately downstream of the enzymatic hydrolysis reactor. Such a measurement involves the advantage that it gives results indicative of the immediate outcome of the hydrolysis step once completed. Similar to above, performing an FTIR measurement on the contents of an enzymatic hydrolysis reactor may mean performing the measurement on the contents of a process stream immediately downstream of any of the enzymatic hydrolysis reactors in the process, or in any combination of these.

Reference designator 305 in FIG. 3 illustrates how, as an addition or alternative to the FTIR measurements 302 and 303, the method may comprise performing an FTIR measurement on a liquid output of a separation step 304 downstream from an enzymatic hydrolysis reactor. The separation step 304 has the purpose of separating solids from liquids.

If the process has the general organization of FIG. 2, there are the first and second solid/liquid separation steps 203 and 206 from which the C6 carbohydrates are collected as process outputs. Additionally there may be further solid/liquid separation steps, from which the liquid fraction is circulated back into earlier steps of the process (for example to step 201 or to step 204). An FTIR measurement like that of reference designator 305 has a slightly different purpose depending on where in the overall process the separation step 304 is located and what is its purpose. If the outcome of any of the first or second steps 203 or 206 in FIG. 2 is measured, the purpose it to ensure that as much C6 carbohydrates as possible are obtained. On the other hand, if the outcome of any subsequent separation steps is measured, the purpose is to ensure that there are as little C6 carbohydrates left in the liquid fraction as possible. Otherwise the previous steps would have been unsuccessful or at least suboptimal in directing the desired C6 carbohydrates towards the output of the process.

Any of the FTIR measurements described above may be performed with a dedicated FTIR measurement device installed at the corresponding point in the process. Such a distributed measurement strategy involves the advantage that the FTIR measurements may be continuous or at least freely timed at any point of the process, and/or several FTIR measurements may be performed in parallel at different steps in the process. FIG. 4 illustrates an alternative approach in which the method comprises controllably conveying samples taken from a plurality of sampling points along the manufacturing process into a common FTIR measurement point 401 in a time-divided manner. FTIR measurements 402 of such plurality of samples may then be performed sequentially at the FTIR measurement point 401.

From each sampling point there is a controllable fluid connection, illustrated schematically in FIG. 4 with a conduit and valve, to the FTIR measurement point 401. Flushing connections 403 and 404 are provided to ensure that the FTIR measurement point 401 can be cleaned of the remains of the previous sample before a next sample comes in. The controllable fluid connections may be manually operable and/or there may be an automatic control system capable of controlling the sampling and measuring sequence.

The approach of FIG. 4, i.e. controllably conveying samples to a common measurement point, involves the advantage that only one FTIR measurement apparatus (or at least a smaller number of FTIR measurement apparatuses) is needed. This leads to corresponding advantages concerning the cost of acquiring and installing the measurement system, as well as simpler maintenance and calibration.

Above it was outlined that the controlling of the manufacturing process of chemical bioproducts involves controlling the value of at least one process parameter based at least partly on the results obtained from at least one FTIR measurement. According to an embodiment, such controlling comprises controlling the dosing of at least one enzyme into one or more enzymatic hydrolysis step in the process.

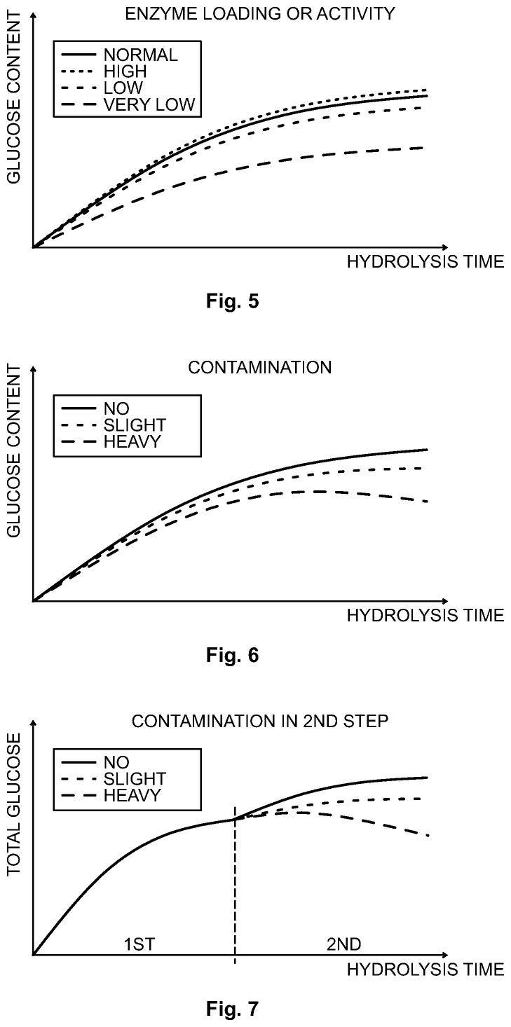

FIG. 5 illustrates an example how the loading or activity of an enzyme (or enzyme combination) may affect the proceeding of the hydrolysis reaction. The horizontal axis represents the residence time of a batch of slurry in an enzymatic hydrolysis reactor, and the vertical axis represents the glucose content of the slurry. It is typical to the hydrolysis reaction that the glucose content begins to increase relatively rapidly, but the increasing slows down or even levels out as the reaction approaches a balance state. The rate of increase in the glucose content, as well as the final level that can be achieved, both may depend on the loading or activity of the enzyme (or enzyme combination). As the enzymes are relatively expensive, it is not advisable to use them in excess. On the other hand, too low loading leads to suboptimal yield of glucose. If there is an FTIR measurement with which the development of the glucose content within a batch of slurry can be monitored, this may help in deciding, whether some enzyme should be still added or whether the exact constitution of an enzyme combination should be tuned for the currently processed batch.

It is not necessary, however, to have the FTIR measurement directed to the actual contents of an enzymatic hydrolysis reactor to make decisions about enzyme loading or activity. In other words, controlling the dosing of enzymes does not need to be based on the FTIR measurement 302 shown in FIG. 3. Similar decisions may be made regarding a subsequent batch based on the results that were obtained from a previous batch, for example with FTIR measurements such as 303 or 305 in FIG. 3.

Additionally or alternatively, the controlling of the value of the process parameter may comprise controlling the residence time of the processed product in the enzymatic hydrolysis. Similar to the controlling of enzyme loading or activity, decisions about the residence time may concern the current batch if there is an FTIR measurement in the reactor (like measurement 302), and/or they may concern a subsequent batch if there are one or more FTIR measurements downstream from the reactor (like measurements 303 and 305).

FIGS. 6 and 7 show examples of how microbial contamination in the slurry may affect the development of the glucose content. FIG. 6 shows how the FTIR measurements—which give an indication of the glucose content—may indicate contamination in an individual enzymatic hydrolysis step, and FIG. 7 shows how corresponding measurements may indicate contamination in a second enzymatic hydrolysis step downstream from a first enzymatic hydrolysis step in the process. Microbial contamination has typically the effect that the unwanted microbes begin to consume the glucose that was already obtained through hydrolysis. This may mean that the glucose content increases slower than it should, as illustrated by the middle graph in FIG. 6 and the middle one of the branching graphs in FIG. 7. If the microbial contamination is serious, it may even mean that the glucose content may begin to drop, as illustrated by the lowermost graph in FIGS. 6 and 7.

In case the FTIR measurement gives indications about microbial (or chemical) contamination, the consequent controlling of the value of a process parameter may comprise controlling the efficiency of intermediate cleaning in preparation of a subsequent product batch in the manufacturing process. The term cleaning-in-place or the corresponding acronym CIP are frequently used to mean such intermediate cleaning of processing equipment.

Chemical constituents in the slurry that are unwanted but may still occur involve at least furfural, carboxylic acid, lactic acid, acetic acid, and ethanol. Additionally, there may be chemical constituents the occurrence of which may be difficult to predict but that may become detectable as anomalous spectral features in one or more of the FTIR measurements. The controlling of the value of a process parameter may involve e.g. directing a batch to reject or at least cutting short its further processing if it is found to contain excessive amounts of such an unwanted chemical constituent.

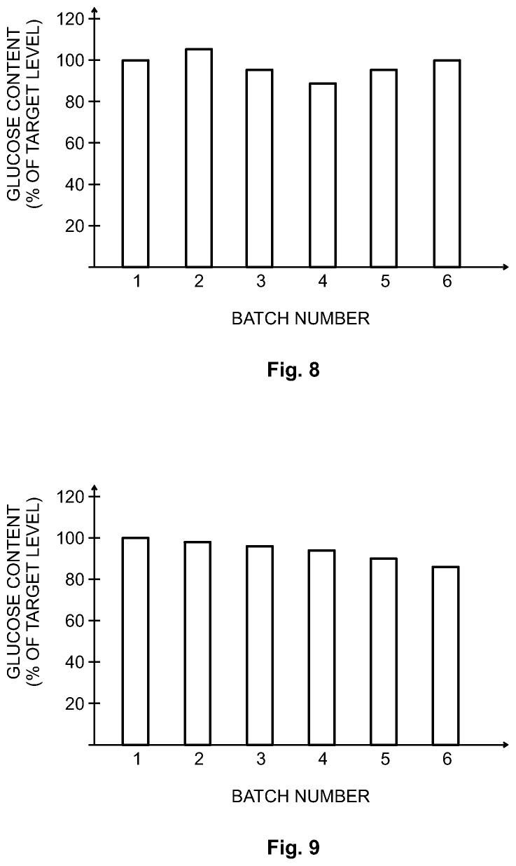

FIGS. 8 and 9 show examples of how the decision-making process may utilize FTIR measurements that only become available after the enzymatic hydrolysis of a batch has been completed (like FTIR measurements 303 or 305 in FIG. 3). FIG. 8 shows how the obtained glucose content appears to show random variation from batch to batch, while in FIG. 9 there is a worrisome decreasing trend in the obtained glucose content that becomes lower and lower. In the case of FIG. 8, if there were no changes in the process parameter values, the root cause of the variation may be e.g. variation in the raw material and/or variation in how the preceding steps succeeded in the manufacturing process of chemical bioproducts. Information about the variation may be fed back to earlier steps in the manufacturing process, where they may be correlated against what is known about the possibly varied aspects there, possibly leading to corrective action. In the case of FIG. 9, one apparent reason behind the worrisome trend is again microbial contamination, because—at least in the absence of sufficient cleaning between batches—it is typical to microbe populations that they continue growing, causing more and more disadvantageous consequences. Findings like those in FIG. 9 could lead e.g. to a decision to perform a more thorough cleaning of the reactors before taking in the next batch.

The method may involve utilizing artificial intelligence in making the decisions about process parameter values on the basis of the FTIR measurement(s). A decision-making controller of the process may collect data about previously used values of process parameters and the corresponding FTIR measurement results and make conclusions about trends and interrelationships that might be difficult or impossible to perceive with only human intelligence. Such a decision-making controller that is arranged to utilize artificial intelligence may then develop further and extrapolate from initial, basic control algorithms to make decisions about process parameters that most optimally meet each available FTIR measurement result of any future batch to be processed.

Above, the viewpoint has been mostly that of a method. From an apparatus viewpoint, there is provided an arrangement for controlling enzymatic hydrolysis in a manufacturing process of chemical bioproducts. The arrangement comprises at least one reactor for subjecting a process stream of said manufacturing process to enzymatic hydrolysis. The reactor may have the general appearance of a vessel or large pipe through which the process stream goes. In a batch-wise process, consecutive batches of the processed product are each held in a reactor vessel for a certain reaction time, while in a continuous process, the processed product may be made to slowly flow through a pipe-formed reactor, along which the enzymatic hydrolysis takes place.

The arrangement comprises additional processing equipment upstream and downstream of the reactor, where “upstream” and “downstream” are defined by the general flowing direction of the processed product in the process. Such additional processing equipment may comprise for example channels, pipes, pumps and conveyors; further reactors; decanters and filtering equipment; mixing equipment; and the like. Defining that some piece of the additional processing equipment is located upstream and downstream of the reactor does not mean immediately before or immediately after the reactor, but there may be other equipment therebetween.

The arrangement comprises at least one FTIR measurement station configured to measure the content of one or more carbohydrates in a process fluid contained in a reactor or in any of the additional processing equipment. Such an FTIR measurement station typically comprises the probe or measurement head, optical equipment for directing the infrared radiation to and from the probe, and electronic processing means capable of generating and detecting the infrared radiation and of converting the raw measurement data into a form in which it constitutes spectral information usable and understandable by a process controller that is coupled to receive measurement results from the (at least one) FTIR measurement station.

The process controller is configured to control the value of at least one process parameter of the manufacturing process at least partly on the basis of the received measurement results. The purpose of such controlling of the parameter value(s) is to affect the conversion of cellulose and hemicellulose into monomeric carbohydrates and/or the relative content of soluble lignin in relation to monomeric carbohydrates in the enzymatic hydrolysis.

One possibility to arrange the hardware for the FTIR measurement(s) is to make the arrangement comprise a plurality of FTIR measurement stations, each configured to measure the content of the respective carbohydrate or carbohydrates in a respective process fluid. Another possibility is to make the arrangement comprise a common FTIR measurement station, so that fluid handling means are configured to controllably convey samples taken from a plurality of sampling points along said manufacturing process into said common FTIR measurement point in a time-divided manner. The use of these two possibilities has been explained in more detail earlier under the method viewpoint.

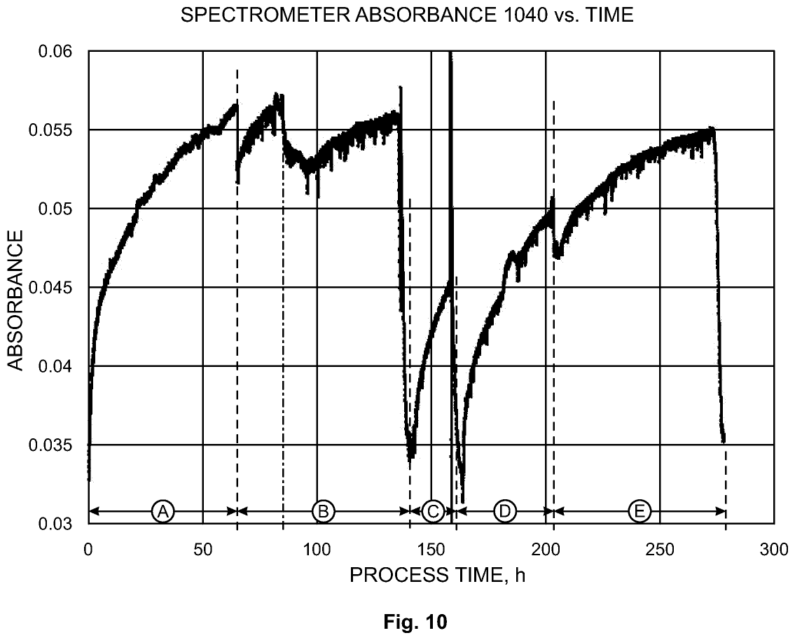

FIGS. 10 to 13 illustrate the applicability of FTIR measurements for determining glucose content of slurry during enzymatic hydrolysis. For producing these graphs, five series of measurements A to E were made. Each of these involved subjecting a batch of pretreated process material (outcome of a pretreatment process of the kind explained above with reference to the pretreatment part of FIG. 1) to enzymatic hydrolysis. Measurement series A, B, and C were made from batches subjected to single-step enzymatic hydrolysis, while measurement series D-E were made from a batch subjected to two consecutive steps of enzymatic hydrolysis. Of the last-mentioned, measurement series D shows the measurement results during the first step and measurement series E shows the measurement results during the second step respectively. The measurement series are here graphically shown as following each other on the time axis, but this is just a way of showing the results graphically: except for the two-step nature of the measurement series D-E mentioned above, the individual measurement series were independent of each other.

Each individual FTIR measurement gives an absorbance value for each wave number in the wave number range involved. Plotting these absorbance values on a wave number (or wavelength) axis gives a momentary FTIR spectrum. By performing repeated FTIR measurements while the hydrolysis reaction proceeds, a time series of the wave-number-specific absorbance values can be accumulated.

FIG. 10 shows the time series of the absorbance values that the FTIR measurement device gave for the wave number 1040 l/cm in each measurement series A to E. Absorbance at wave number 1040 has been found to correlate relatively well with glucose content. FIG. 10 confirms this finding, by showing a generally increasing trend in absorbance at wave number 1040 towards the end of each measurement series.

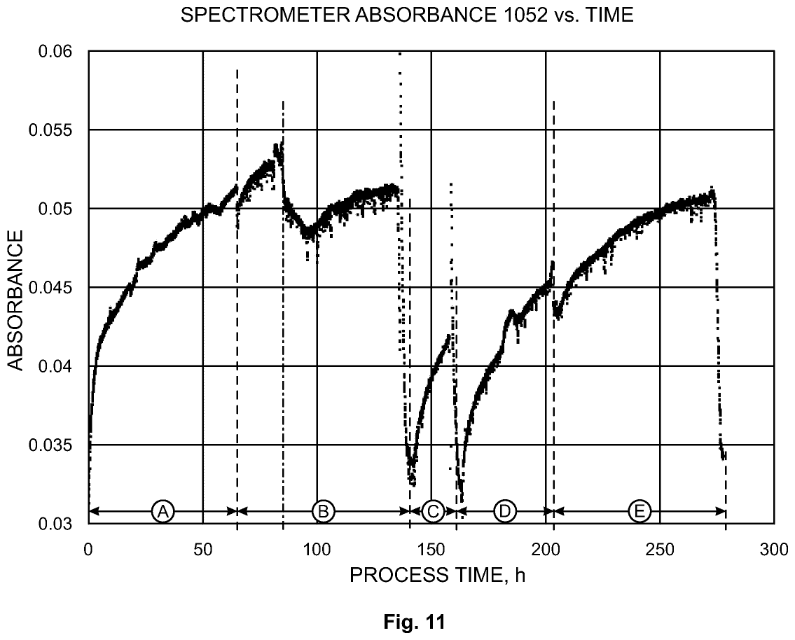

FIG. 11 shows the time series of the absorbance values that the FTIR measurement device gave for the wave number 1052 l/cm in each measurement series A to E. Similar to wave number 1040, also absorbance at wave number 1052 has been found to correlate relatively well with glucose content. FIG. 11 confirms this finding, by showing a generally increasing trend in absorbance at wave number 1052 towards the end of each measurement series.

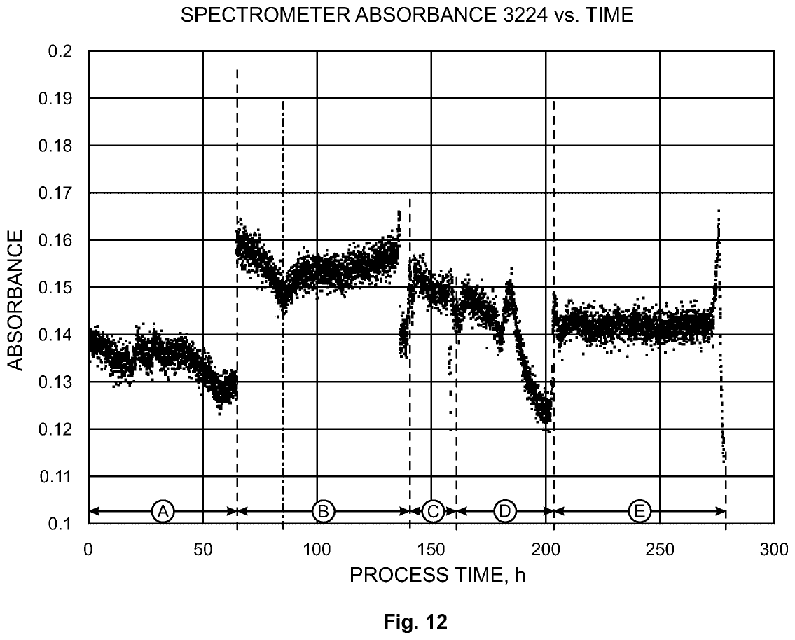

It has been found that there are some wave numbers within the range 648-4000 l/cm that are applicable for temperature compensation in the FTIR measurement. For example, the FTIR-measured absorbance for 3224 l/cm is relatively insensitive to such changes in chemical constitution that occur during enzymatic hydrolysis. Instead, absorbance at 3224 l/cm has been found to change as a function of the temperature of the slurry. FIG. 12 shows the time series of the absorbance values that the FTIR measurement device gave for the wave number 3224 l/cm in each measurement series A to E.

As similar, temperature-dependent changes can be expected to appear also at those wave numbers that are indicative of chemical constitution, one may use the measured absorbance at 3224 l/cm (and/or other wave numbers found to be appropriate for this purpose) to mitigate temperature-induced inaccuracy. The basic principle of such mitigating involves calculating, for each individual FTIR spectrum, a correction factor on the basis of the absorbance value(s) at the temperature-indicative wave number(s) and summing that correction factor to the measured absorbance value(s) at the chemical-constitution-indicative wave number(s).

Findings of the kind explained above make it possible to construct a calculation model, in which one uses the FTIR-measured absorbance values for selected wave numbers to produce indications of glucose content in the slurry. An example of a general form of such a calculation model is

$${C}_{gluc} ( t ) = {\sum }_{i = 1}^{N} {α}_{i} {A}_{i} ( t ) + {\sum }_{j = 1}^{M} {β}_{j} {A}_{j} ( t ) + Γ$$

where C_{gluc}(t) is the concentration of glucose at time t,
aᵢ is a constant weight for the i:th term in the first sum,
Aᵢ(t) is the measured absorbance value at the i:th wave number
N is the total number of wave numbers at which the FTIR measurement shows significant dependency on glucose content,
βⱼ is a constant weight for the j:th term in the second sum,
Aⱼ(t) is the measured absorbance value at the j:th wave number,
M is the total number of wave numbers at which the FTIR measurement shows dependency on temperature only, and
Γ is a constant.

In other words, the formula above represents a calculation in which one gives weights αᵢ to all those wave numbers that are used to examine glucose content, and weights βⱼ to all those wave numbers that are used to compensate for changes in temperature. The first term on the right in the formula represents summing the weighted contributions of all those N wave numbers that are indicative of glucose content. The second term on the right in the formula represents temperature compensation that takes into account the weighted contributions of all those M wave numbers that are indicative of temperature.

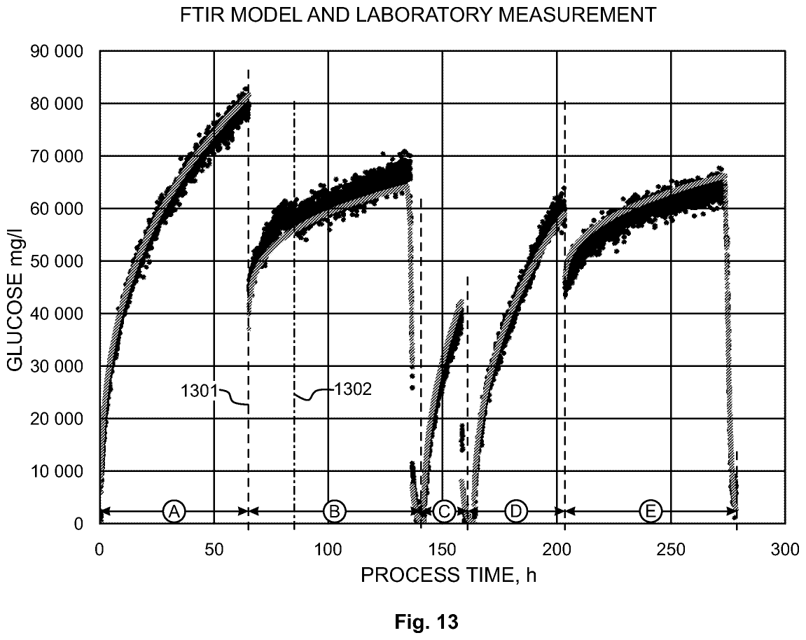

FIG. 13 illustrates a comparison where batches of slurry were subjected to enzymatic hydrolysis, the proceeding of which was monitored with repeated FTIR measurements in the wave number range 648-4000 l/cm. Glucose content was calculated as a function of time by using the formula above with parameter values N=2, M=1, Γ=49779, α₁=11120653, α₂=−9320009, and β₁=162864. The two wave numbers to contribute to the first sum were 1040 l/cm (i=1) and 1052 l/cm (i=2), and the only wave number to contribute to the second sum was 3224 l/cm (j=1). For each moment of time t, the value obtained from the formula was plotted as a black spot. Simultaneously with the FTIR measurements, samples were taken of the slurry and their glucose content was measured with laboratory methods. The grey curves represent a best mathematical fit of a smooth curve to the laboratory measurements.

FIG. 13 shows a relatively good agreement between the “clouds” of black spots and the grey curves. This proves that with even a relatively coarse calculational model one can conclude the glucose content of the slurry relatively accurately from the FTIR measurement. The calculational model can be made better by increasing the numbers N and M, i.e. finding more wave numbers at which the FTIR-measured absorbance is indicative of either the chemical constitution or temperature, and defining appropriate weights αᵢ and βⱼ. The last-mentioned can be made by statistical or chemometric methods, i.e. by comparing the calculated results to laboratory measurements and selecting the weight values that give the best match for example in the sense of least sum of squares.

An interesting detail in FIG. 13 is seen around the transition between measurement series A and B (see point 1301) and a subsequent point 1302 about one third into the duration of measurement series B. In both FIGS. 10 and 11, between the vertical dash- and dash-dot lines that mark the corresponding points in time, the measured absorbance at 1040 l/cm and 1052 l/cm continues to increase after a brief drop, as if the first measurement series A was still going on. However, FIG. 12 shows how a significant transient increase takes place in measured absorbance at 3224 l/cm at point 1301, after which a subsequent decrease takes place between said vertical dash- and dash-dot lines. In other words, the process temperature has changed sharply at the transition point 1301 between measurement series A and B, followed by a continuous change. A decreasing temperature increases absorbance at 1040 l/cm and 1052 l/cm but decreases absorbance at 3224 l/cm. As is seen in FIG. 13, using absorbance at 3224 l/cm (and/or at any other wave number that is a good, chemical-constitution-independent indicator of temperature) to compensate for temperature-based inaccuracies produces a significant improvement in using the FTIR measurement to determine the glucose content during enzymatic hydrolysis.

It is obvious to a person skilled in the art that with the advancement of technology, the basic idea of the invention may be implemented in various ways. The invention and its embodiments are thus not limited to the examples described above, instead they may vary within the scope of the claims.

1. A method for controlling enzymatic hydrolysis in a manufacturing process of chemical bioproducts, the method comprising:
performing at least one Fourier Transform Infrared, later FTIR, measurement on at least one process fluid of said manufacturing process, and
controlling the value of at least one process parameter based on the results obtained from said at least one FTIR measurement;
wherein said results indicate the content of one or more carbohydrates in the respective process fluid, and wherein said controlling of the value of the process parameter is performed in order to affect at least one of: the conversion of cellulose and hemicellulose into monomeric carbohydrates in said enzymatic hydrolysis, the relative content of soluble lignin in relation to monomeric carbohydrates in said enzymatic hydrolysis.

2. A method according to claim 1, wherein:
the method comprises performing an FTIR measurement on the contents of an enzymatic hydrolysis reactor where said enzymatic hydrolysis is currently taking place.

3. A method according to claim 2, comprising:
taking a sample of the contents of said enzymatic hydrolysis reactor, and
conveying said sample into an FTIR measurement point for performing said FTIR measurement.

4. A method according to claim 1, wherein:
the method comprises performing an FTIR measurement on the contents of a process stream immediately downstream of said enzymatic hydrolysis reactor.

5. A method according to claim 1, wherein:
the manufacturing process comprises a separation step downstream of said enzymatic hydrolysis reactor for separating solids from liquids, and
the method comprises performing an FTIR measurement on a liquid output of said separation step.

6. A method according to claim 1, comprising:
controllably conveying samples taken from a plurality of sampling points along said manufacturing process into a common FTIR measurement point in a time-divided manner, and
performing FTIR measurements of said plurality of samples sequentially at said FTIR measurement point.

7. A method according to claim 1, wherein said controlling of the value of the process parameter comprises controlling the dosing of at least one enzyme into said enzymatic hydrolysis.

8. A method according to claim 1, wherein said controlling of the value of the process parameter comprises controlling the residence time of the processed product in said enzymatic hydrolysis reactor.

9. A method according to claim 1, wherein:
said enzymatic hydrolysis is made in consecutive product batches in the process, and
said controlling of the value of the process parameter comprises controlling the efficiency of intermediate cleaning in preparation of a subsequent product batch in said manufacturing process.

10. A method according to claim 1, wherein:
said results of the FTIR measurement are obtained by calculating, for a plurality of instants of time, a respective weighted linear combination of FTIR-measured absorbance values at selected wave numbers and using the calculated weighted linear combination as an indicator of measured concentration of a monomeric carbohydrate at each such instant of time.

11. A method according to claim 10, wherein said selected wave numbers include at least one compensatory wave number selected for temperature compensation, absorbance at said compensatory wave number being less sensitive to the concentration of said monomeric carbohydrate than absorbances at others of said selected wave numbers.

12. An arrangement for controlling enzymatic hydrolysis in a manufacturing process of chemical bioproducts, the arrangement comprising:
at least one reactor for subjecting a process stream of said manufacturing process to enzymatic hydrolysis,
additional processing equipment upstream and downstream of said reactor in the process,
at least one Fourier Transform Infrared, later FTIR, measurement station configured to measure the content of one or more carbohydrates in a process fluid contained in said reactor or in any of said additional processing equipment, and
a process controller coupled to receive measurement results from said at least one FTIR measurement station,
wherein said process controller is configured to control the value of at least one process parameter of the manufacturing process at least partly on the basis of the received measurement results in order to affect at least one of: the conversion of cellulose and hemicellulose into monomeric carbohydrates in said enzymatic hydrolysis, the relative content of soluble lignin in relation to monomeric carbohydrates in said enzymatic hydrolysis.

13. An arrangement according to claim 12, comprising a plurality of FTIR measurement stations, each configured to measure the content of the respective carbohydrate or carbohydrates in a respective process fluid.

14. An arrangement according to claim 12, comprising a common FTIR measurement station, so that said fluid handling means are configured to controllably convey samples taken from a plurality of sampling points along said manufacturing process into said common FTIR measurement point in a time-divided manner.

15. Use of a Fourier Transform Infrared, later FTIR, measurement, made on at least one process fluid of a manufacturing process of chemical bioproducts, to control the value of a process parameter based on the results obtained from said FTIR measurement;
wherein said results indicate the content of one or more carbohydrates in the respective process fluid, and wherein said controlling of the value of the process parameter is performed in order to affect at least one of: the conversion of cellulose and hemicellulose into monomeric carbohydrates in said enzymatic hydrolysis and/or the relative content of soluble lignin in relation to monomeric carbohydrates in said enzymatic hydrolysis.
